(19) United States
(12) Patent Application Publication
(10) Pub. No.: US 2010/0114328 A1
(43) Pub. Date: May 6, 2010

(54) RESORBABLE MEMBRANE IMPLANTING METHODS FOR REDUCING ADHESIONS

(76) Inventors: Michael T. Milbocker, Holliston, MA (US); Lukas Bluecher, Eurasburg (DE)

Correspondence Address: STOUT, UXA, BUYAN & MULLINS LLP, 4 VENTURE, SUITE 300, IRVINE, CA, 92618, US

(21) Appl. No.: 12/498,291

(22) Filed: Jul. 6, 2009

Related U.S. Application Data
(63) Continuation-in-part of application No. 10/660,461, filed on Sep. 10, 2003.
(60) Provisional application No. 61/078,431, filed on Jul. 6, 2008.

(51) Int. Cl. A61F 2/02; A61B 19/08

(52) U.S. Cl. 623/23.72; 128/849

(57) ABSTRACT

A resorbable thin membrane is applied over a treatment site before a treatment is applied over the resorbable thin membrane to the site. In a particular implementation, a resorbable thin membrane is adhesively applied over a treatment site of tissue before a treatment is conducted onto the tissue whereby the treatment is performed through the resorbable thin membrane. The treatment can be an incision that is made through both the resorbable membrane and into or through the tissue.

### CROSS-REFERENCE TO RELATED APPLICATIONS

This application claims the benefit of U.S. Provisional Application No. 61/078,431, filed Jul. 6, 2008 and entitled Resorbable Membrane Implanting Methods for Reducing Adhesions (Att. Docket MB8134PR) and is a continuation-in-part of U.S. application Ser. No. 10/660,461, filed Sep. 10, 2003 and entitled Methods of Promoting Enhanced Healing of Tissues After Cardiac Surgery (Att. Docket MA9758P), the entire contents of both which are expressly incorporated herein by reference.

This application is related to U.S. application Ser. No. 12/480,655, filed Jun. 8, 2009 (Att. Docket MB8110P); U.S. application Ser. No. 10/019,797, filed Jul. 26, 2002 (Att. Docket MB9962P); U.S. application Ser. No. 10/385,399, filed Mar. 10, 2003 (Att. Docket MA9496CON), now U.S. Pat. No. 6,673,362, the contents of all which are expressly incorporated herein by reference. This application is also related to U.S. application Ser. No. 10/631,980, filed Jul. 31, 2003 (Att. Docket MA9604P); U.S. application Ser. No. 11/203,660, filed Aug. 12, 2005 (Att. Docket MB9828P); and U.S. application Ser. No. 12/199,760, filed on Aug. 27, 2008 (Att. Docket MB8039P). The foregoing applications are commonly assigned and the entire contents of all of them are expressly incorporated herein by reference.

### BACKGROUND OF THE INVENTION

1. Field of the Invention

The present invention relates generally to medical implants and, more particularly, to methods of applying resorbable membranes for inducing healing, or proper healing, of tissues, such as, for example, parietal pericardium tissues.

2. Description of Related Art

In the context of tissue repair, adhesions, typically comprising scar tissue, can occur during the initial phases, or in certain instances subsequent phases, of the healing process after surgery, although adhesions may occur as a result of other processes, as well, such as during or following the onset of disease. The formation and/or persistent presence of excessive or unacceptable amounts of scar tissue, or fibrosis, can present itself as an important or even central issue in medicine. Scar tissue can in several instances block arteries, immobilize joints, damage internal organs, and can in some instances generally or partially impede a body's ability to maintain vital functions. Every year, about 1.3 million people are hospitalized due to the damaging effects of fibrosis. Fibrosis can follow surgery in the form of adhesions, keloid tumors or hypertrophic (very severe) scarring. Fibrotic growth can also proliferate and invade the healthy tissue that surrounds it, even after the original injury heals. Too much scar tissue may cause physiological roadblocks that in severe cases can disfigure, cripple or even kill.

An important pathology in which fibrosis can be particularly problematic is cardiac surgery. The number of patients undergoing cardiac surgery has been steadily increasing, and as a consequence, the number of cardiac complications has also increased. One of the more common forms of adhesions in this context can occur after the cardiac surgery as a direct result of the current or a prior cardiac surgical intervention.

Cardiac surgical procedures can often be associated with unique conditions of adhesion, which can begin to form immediately following the surgical procedure. Such conditions can persist often times with lower rates of treatment success as compared to similar complications of other tissues. Despite the need for effective treatments, doctors typically have few optimal remedies to help them control this relatively prevalent condition.

In addition to being relatively prevalent, such conditions have proven themselves according to a growing body of data to be relatively persistent. That is, in the context of cardiac surgical procedures, for example, scar tissue treatments may be less effective in preventing or attenuating fibrosis than with other procedures.

For instance, thin-membrane implantation measures for attenuating adhesion following cardiac surgery, such as disclosed in U.S. application Ser. No. 10/660,461, may in some instances and ways be less effective than similar procedures or interventions taken in connection with other tissues and/or other parts of the body, potentially as a consequence of the continuous flexing and moving of the cardiac tissue. Following a cardiac surgical procedure, for example, the careful placement of a thin resorbable membrane over one or more vicinities of tissue affected by the procedure (e.g., over one or more areas, apertures, protuberances, or edges, which were created by, affected by, or which just underwent, an incision) may not be altogether effective in providing an optimum or even adequate attenuation of tissue adhesions in that vicinity, again potentially as a result of a relatively excessive amount, or nature, of movements of or near the affected tissue in need of treatment.

There continues to be a need for improved methods of inducing proper tissue healing, e.g. healing without adhesion or with reduced rates of adhesion, for relatively non-static tissues affected by surgical interventions such as open heart surgery.

### SUMMARY OF THE INVENTION

The present invention provides resorbable thin membranes for use in promoting healing of tissues, such as, as a primary point of interest, non-static (e.g., frequently or constantly moving) tissues, which may be affected, for example, by surgical interventions, such as open heart surgery, and methods for using the same.

According to one feature of the present invention, a resorbable thin membrane is applied over a treatment site before a treatment is applied thereto. According to one feature of the invention, the treatment can then be performed through the resorbable thin membrane. In a particular implementation, a resorbable thin membrane is adhesively applied over a treatment site of tissue before a treatment is conducted onto the tissue whereby the treatment is performed through the resorbable thin membrane. The treatment can be an incision, whereby the incision can be made through both the resorbable membrane and into (e.g., through) the tissue. The resorbable thin membranes are used according to the technique to promote the healing of tissues, such as cardiac tissues, which may be affected, for example, by surgical interventions.

While the apparatus and method has or will be described for the sake of grammatical fluidity with functional explanations, it is to be expressly understood that the claims, unless indicated otherwise, are not to be construed as limited in any way by the construction of “means” or “steps” limitations, but are to be accorded the full scope of the meaning and equivalents of the definition provided by the claims under the judicial doctrine of equivalents.

Any feature or combination of features described or referenced herein are included within the scope of the present invention provided that the features included in any such combination are not mutually inconsistent as will be apparent from the context, this specification, and the knowledge of one skilled in the art. In addition, any feature or combination of features described or referenced may be specifically excluded from any embodiment of the present invention. For purposes of summarizing the present invention, certain aspects, advantages and novel features of the present invention are described or referenced. Of course, it is to be understood that not necessarily all such aspects, advantages or features will be embodied in any particular implementation of the present invention. Additional advantages and aspects of the present invention are apparent in the following detailed description and claims that follow.

### DETAILED DESCRIPTION OF THE PRESENTLY PREFERRED EMBODIMENTS

Reference will now be made in detail to the presently preferred embodiments of the invention. Although the disclosure herein refers to certain illustrated embodiments, it is to be understood that these embodiments are presented by way of example and not by way of limitation. The intent accompanying this disclosure is to discuss exemplary embodiments with the following detailed description being construed to cover all modifications, alternatives, and equivalents of the embodiments as may fall within the spirit and scope of the invention as defined by the appended claims. It is to be understood and appreciated that the process steps and structures described and referenced herein do not cover a complete process flow for the manufacture of the disclosed and referenced structures. The present invention may be practiced in conjunction with various integrated circuit fabrication and other techniques that are conventionally used in the art, and only so much of the commonly practiced process steps are included herein as are necessary to provide an understanding of the present invention.

A resorbable thin membrane is applied over a treatment site before a treatment is applied thereto. According to one feature of the invention, the treatment can then be performed through the resorbable thin membrane. In a particular implementation, a resorbable thin membrane is adhesively applied over a treatment site of tissue before a treatment is conducted onto the tissue whereby the treatment is performed through the resorbable thin membrane. The treatment can be an incision, whereby the incision can be made through both the resorbable membrane and into (e.g., through) the tissue. The resorbable thin membranes are used according to the technique to promote the healing of tissues, such as cardiac tissues, which may be affected, for example, by surgical interventions.

In one embodiment, methods of the present invention can comprise the provision and manipulation of a healing membrane to form a patch over, or to otherwise protect or contact in modified embodiments, a tissue before the surgical intervention to induce proper healing of that tissue. The membrane can comprise any membrane described or referenced herein, or can comprise any membrane described or referenced in any document referenced in a document which, itself, is referenced herein.

A preferred, suitable, or contemplated, membrane may comprise, for example, part or all of any one or more of: (a) any membrane, membrane component, or part such as described or referenced herein or in U.S. application Ser. No. 10/019,797, U.S. application Ser. No. 12/480,655, U.S. application Ser. No. 12/199,760, and U.S. application Ser. No. 11/203,660, in any combination; and (b) any item of the immediately preceding group modified or combined in any way as would be recognized to be feasible by one knowledgeable in the art. The healing membranes are preferably constructed from a resorbable, or in some implementations, at least partially resorbable, polymer. The membranes can in preferred implementations be constructed to have one or more of an additional, supplemental, or reinforcing, or additional (a) structure (e.g., ridge, protuberance, fiber, additive, or agent) or (b) layer, special-acting layer, or reinforcing layer.

The formation, or provision, of the membrane with the tissue to be treated (e.g., protected), can comprise, for example, contacting (e.g., placement) of the membrane onto tissue to form, for example, a patch over the tissue. The contacting can be performed on one or more surfaces of the tissue, and can be implemented to generate one or more layers (e.g., overlapping, non-overlapping, or partially overlapping) of the membrane.

For example, membranes can be secured on an outer or inner surface of an area or volume (e.g., wall) of tissue, or may be disposed on opposing (e.g., inner and outer) surfaces of a tissue to be contacted, in one or more (e.g., contacting one another) structures or layers.

The membrane can be contacted with the tissue according to any one or more of being frictionally secured, secured by operation of a “sticky” property of the membrane (c.f., U.S. application Ser. No. 10/019,797, U.S. application Ser. No. 12/480,655, U.S. application Ser. No. 12/199,760) wherein for example the membrane is secured by operation of an adhesive or other intervening substance (e.g., disposed on or between at least a part of one or more of the tissue and the membrane), sutured, heat welded, secured using any securing technique described or referenced herein or any document referenced in any document referenced herein, or in modified implementations secured by other known securing means, in any combination or permutation. An example can comprise the provision of a membrane, which is formed, loaded or coated with an adhesive-type agent to provide a “sticky” property wherein, for instance, a surface of the membrane may be coated or loaded with a surface modifier as described, for example, in U.S. application Ser. No. 10/019,797, to provide a slightly sticky (e.g., approximating that of a Post-It®) characteristic.

The surgical intervention (i.e., that being performed on the cardiac tissue and being responsible for causing the adhesions) may comprise contacting, or disrupting the tissue. According to the present invention, the membrane is formed (e.g., placed) or disposed in a vicinity of (e.g., contacted with) or disposed in connection with (e.g., over) the tissue to be disrupted, before the surgical intervention. The disruption of the tissue is then performed. Disrupting of the tissue can comprise forming an opening into the tissue, cutting the tissue, incising the tissue (e.g., forming an incision in the tissue to access an interior of the heart), or otherwise ablating or treating (e.g., changing one or more physical characteristics of) the tissue.

In a typical implementation, the surgical intervention comprises, following placement of the patch, cutting or incising through, on, or at least partially into, the tissue to be treated, in a direct area of (or, alternatively, in a vicinity of or adjacent to) the patch. For instance, the surgical intervention can comprise cutting or incising through both the patch and the tissue following placement of the patch onto the tissue (e.g., causing the two to be joined, e.g., intimately or in a way in which they are touching) together by suturing and/or adhesive means. The tissue can comprise cardiac, or in modified embodiments other types of, tissue.

According to one feature of the present invention, the tissue can comprise a tissue that is in need of reinforcement and/or will be in need of reinforcement following treatment. For example, the tissue can comprise (I) a damaged or disrupted layer, surface or membrane that is in need of reinforcement and/or (II) a layer, surface or membrane that will be cut, lacerated, modified (e.g., by a laser procedure) or otherwise weakened in a treatment rendering it in need of reinforcement following the treatment. In a preferred embodiment, the tissue can comprise a type II implementation in the form of a membrane (e.g., a conjunctiva or pericardium) which will be compromised/damaged, as known in the art, during a treatment procedure. Upon placement (e.g., affixation by adhesion) of a patch over the treatment site and (e.g., thereafter) a treatment in, for example, a type II scenario, closing of the treatment site (e.g., that has been cut open) and/or repositioning of the membrane (e.g., that is damaged from the initial dispositioning, and/or that will be damaged from repositioning, of the membrane back to a pre-treatment position orientation, or state, such as repositioning of the membrane back over the treatment site) can be accomplished with the membrane and the patch effectively forming a single layer. The layer (e.g., which has been compromised/weakened/disrupted) can be secured (e.g., an opening therethrough closed, a tear closed or mended, or a position/orientation/state set) with any technique described or referenced herein (e.g., with sutures, tacks, staples, tabs and/or slots, adhesive, and/or heat welding).

Following disruption of the patch and/or the tissue, and, typically, following an ensuing surgical intervention (e.g., a known open-heart surgical procedure), the area of disruption (e.g., the incision) can be treated, mended, or otherwise affected to promote, for example, healing. For example, following formation of an incision through the patch and tissue, and following a surgical procedure within or near the tissue, the opening (comprising the tissue and the patch) can be secured (e.g., closed) using components or agents including sutures, tacks, staples, tabs and/or slots, adhesives, tissue welding, or other means. In the instance of suturing an opening to close it, for example, and typically in other similar instances, the thin membrane is affixed to the tissue (e.g., the pericardium), so that the tissue and the membrane effectively form a single layer, which is closed (e.g., with sutures). Any of the securing can be performed with parts (e.g., edges) of the patch specially formed (e.g., at a time of manufacture, or immediately pre-surgery; e.g., with thicker regions and/or holes and/or tabs and/or slots and/or regions of greater strength such as by stronger polymer or reinforcement fibers) such as described and referenced herein (e.g., as in U.S. application Ser. No. 10/631,980 or U.S. application Ser. No. 11/203,660).

Additionally, or alternatively, or in any combination, sutures may be implemented along opposing edges to close openings, tears, or gaps in the layer. In a preferred implementation, the sutures are positioned and used in the same, or similar, way that a shoestring draws opposing edges of a shoe together, using any technique and structure known to those in the relevant or analogous art.

Following any such securing or closure, according to one embodiment, additional anti-adhesive implements, such as one or more additional thin membranes as described or referenced herein, may be applied over any part or all of the area, using any disposition or attachment technique and/or agent/component/device described or referenced herein.

Corresponding or related structure and methods described in any one or more of the above-referenced applications and/or any one or more of U.S. application Ser. No. 12/481,302, filed Jun. 9, 2009 (Att. Docket MB8111CIP) and U.S. application Ser. No. 12/481,311, filed Jun. 9, 2009 (Att. Docket MB8112CIP) are incorporated herein by reference in their entireties, wherein such incorporation includes corresponding or related structure (and modifications thereof) which may be, in whole or in part, (i) operable and/or constructed with, (ii) modified by one skilled in the art to be operable and/or constructed with, and/or (iii) implemented/made/used with or in combination with, any part(s) of the present invention according to this disclosure, that of the application and references cited therein, and the knowledge and judgment of one skilled in the art.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. A method comprising adhesively applying a resorbable thin membrane over a treatment site of tissue before a treatment is conducted onto the tissue whereby the treatment is performed through the resorbable thin membrane.

2. The method of claim 1 wherein the treatment comprises an incision made through a layer comprising both the resorbable membrane and a membrane of the tissue.

3. The method of claim 2 wherein the resorbable thin membrane is a substantially planar anti-adhesion healing membrane, which is:
(a) substantially-smooth on at least one side;
(b) substantially uniform in composition;
(c) about 10 microns to about 300 microns in thickness;
(d) non-porous;
(e) constructed from a material comprising a resorbable polymer base material selected from one or more of (a) a poly-lactide polymer, (b) a copolymer of lactides, and (c) a poly-lactide polymer and a copolymer of lactides; and
(f) adapted to be resorbed within a period of less than approximately 24 months from an initial implantation of the resorbable thin membrane.

4. The method of claim 2 and further comprising placing a resorbable membrane over the resorbable thin membrane and over the incision.

5. The method of claim 1 and further comprising placing a thin anti-adhesion resorbable membrane over the resorbable thin membrane and over the treatment site following the treatment.

6. The method of claim 1 wherein a thickness of the resorbable thin membrane is between about 100 and 200 microns.

7. The method of claim 1 wherein the resorbable thin membrane is a healing membrane provided in a sterile packaging.

8. The method of claim 7 wherein the step of placing the resorbable thin membrane in a patient is effective to attenuate tissue adhesion.

9. The method of claim 1 further comprising a step of attaching the resorbable thin membrane using stitches, wherein the attaching step comprises lacing a suturing thread through apertures on opposing edges of an opening or gap of the resorbable thin membrane in a manner resembling an arrangement of a shoelace of a shoe, followed by pulling the suturing thread to close the opening or gap.

10. The method of claim 9 further comprising a step of attaching the resorbable thin membrane to a pericardial membrane using stitches.

11. The method of claim 10 wherein the opposing edges have greater thicknesses than other regions of the resorbable thin membrane.

12. The method of claim 11 wherein the attaching step comprises heat bonding the resorbable thin membrane to the pericardial membrane.

13. The method of claim 1 wherein:
the treatment comprises an incision made through a layer comprising both the resorbable membrane and into the tissue; and
the method further comprises a step of attaching the resorbable thin membrane using stitches, the attaching step comprising lacing a suturing thread through opposing edges of an opening or gap of the layer in a manner resembling an arrangement of a shoelace of a shoe, followed by pulling the suturing thread to close the opening or gap.

14. The method of claim 13 wherein the opposing edges have greater thicknesses than other regions of the resorbable thin membrane.

15. The method of claim 13 wherein the attaching step comprises heat bonding the resorbable thin membrane to a pericardial membrane.
